(19) United States
(12) Patent Application Publication
(10) Pub. No.: US 2025/0352136 A1
(43) Pub. Date: Nov. 20, 2025

(54) MEDICAL DEVICE SYSTEMS FOR THERMAL THERAPY

(71) Applicant: Medicool Technologies Inc., Rochester, MN (US)

(72) Inventors: Jeffrey Rynbrandt, Traverse City, MI (US); Paul A. Friedman, Rochester, MN (US); Samuel J. Asirvatham, Rochester, MN (US); Peter James von Alt, St. Petersburg, FL (US); Walter Hughes Quinn, Jacksonville, FL (US)

(21) Appl. No.: 19/085,807

(22) Filed: Mar. 20, 2025

Related U.S. Application Data
(60) Provisional application No. 63/647,956, filed on May 15, 2024.

(51) Int. Cl. A61B 5/00; A61B 5/287; A61B 5/364; A61F 7/00; A61F 7/12; A61M 25/10

(52) U.S. Cl. CPC A61B 5/4836; A61B 5/287; A61B 5/364; A61B 5/6853; A61F 7/12; A61M 25/1002; A61B 2560/0468; A61B 2562/0247; A61B 2562/227; A61F 2007/0056; A61F 2007/126; A61M 2025/1081; A61M 2205/10; A61M 2205/3303; A61M 2205/3327; A61M 2205/3331; A61M 2205/3606; A61M 2205/50; A61M 2210/125; A61M 2230/005; A61M 2230/04

(57) ABSTRACT

Medical device systems and methods can be used for delivering therapy via temperature modulation. For example, this document describes medical device systems and related methods for the treatment of cardiac arrhythmias by delivering cooling to epicardial tissue. In some embodiments described herein, arrhythmias can be treated by an balloon-based system for painlessly terminating arrhythmias. The devices and methods provided herein permit prompt termination of atrial fibrillation almost immediately after an episode begins (to prevent persistence) and are effective irrespective of patient age and comorbidities.

### CROSS-REFERENCE TO RELATED APPLICATIONS

This application claims the benefit of U.S. Provisional Application Ser. No. 63/647,956 filed May 15, 2024. The disclosure of the prior application is considered part of (and is incorporated by reference in) the disclosure of this application.

### BACKGROUND

### 1. Technical Field

This document relates to medical device systems and related methods for delivering therapy via temperature modulation. For example, this document relates to medical device systems and related methods for the treatment of cardiac arrhythmias by delivering cooling to epicardial tissue.

### 2. Background Information

Cardiac arrhythmias occur when there is a change in the rate and/or rhythm of the heartbeat due to changes in the normal sequence of cardiac electrical impulses. Abnormalities of cardiac rate and/or rhythm are associated with substantial morbidity and economic costs. Different types of cardiac arrhythmias include atrial fibrillation, bradycardia, conduction disorders, premature contractions, tachycardias, and ventricular fibrillation.

Among these conditions, atrial fibrillation is the most common arrhythmia encountered in clinical practice, affecting over 6 million Americans. Recently, cardiac arrhythmias have been reported in hospitalized coronavirus disease 2019 (COVID-19) patients, with one study reporting arrhythmias in 44% of individuals with severe illness. Studies have indicated the incidence of atrial fibrillation in the United States will increase to an estimated 12.1 million people in 2030.

Multiple prospective randomized trials have demonstrated the clinical benefit of implantable cardiac defibrillators (ICDs) in saving the lives of at-risk individuals, leading to their wide-spread adoption. A downside associated with ICD therapy, however, is the pain associated with defibrillation, whether shocks are delivered appropriately or inappropriately.

One particular clinical scenario that remains vexing is that of atrial arrhythmias after cardiac and thoracic surgery. The frequency of atrial fibrillation (AF) after cardiac surgery varies depending on the type of procedure. For patients undergoing isolated coronary artery bypass grafting (CABG), the incidence of postoperative AF is approximately 30%. In the case of valve surgery, the incidence increases to around 40%. When CABG is combined with valve surgery, the incidence of postoperative AF is even higher, at about 50%. The majority of AF episodes occur within the first few days after surgery, with a peak incidence between postoperative days 2 and 4.

Postoperative AF is associated with increased morbidity, mortality, and healthcare utilization, and thus, its management is a critical component of postoperative care.

Patients routinely receive chest tubes in the early postoperative period after cardiac and thoracic surgery. If an element at the distal end of a chest tube were able to monitor the cardiac rhythm (potentially shortening ICU stay), anticipate the development of AF via AI analysis, and then painlessly prevent or terminate AF episodes, such a device would have a significant impact in facilitating patient recovery after cardiac and thoracic surgery with reduced AF, which can result in significant economic savings and shortened ICU and hospital stay.

### SUMMARY

This document describes medical device systems and related methods for delivering therapy via temperature modulation. For example, this document describes medical device systems and related methods for the treatment of cardiac arrhythmias (e.g., atrial fibrillation) by delivering cooling to epicardial tissue. It also describes the acquisition of electrical cardiac signals and their use for detection and prediction of atrial fibrillation.

Patients routinely receive chest tubes in the early postoperative period after cardiac and thoracic surgery. If an element at the distal end of a chest tube were able to monitor the cardiac rhythm (potentially shortening ICU stay), anticipate the development of AF via AI analysis, and then painlessly prevent or terminate AF episodes, such a device would have a significant impact in facilitating patient recovery after cardiac and thoracic surgery with reduced AF, which can result in significant economic savings and shortened ICU and hospital stay.

The ion channels responsible for cardiac electrical wavefront propagation are temperature sensitive. As described herein, cooling terminates fibrillation in animal (references) and human (unpublished observation) atrial fibrillation. This disclosure describes an insertable and percutaneously removable tube to offer post surgical drainage, detection and prediction of AF, and its painless prevention and termination.

In one aspect, this disclosure is directed to a medical device that includes a console, a fluid line defining a fluid supply lumen and a fluid return lumen, and a balloon attached or attachable at a distal end portion of the fluid line. The console includes a control system, a pump, a fluid reservoir, and a fluid cooling system.

Such a medical device may optionally include one or more of the following features. The medical device system may also include one or more electrodes coupled to the balloon or the distal end portion of the fluid line. The medical device system may also include one or more electrical wires that are: (i) extending along the fluid line, (ii) connected or connectable to the one or more electrodes, and (iii) connected or connectable to the control system. The control system may be programmed and operable to: (i) analyze EKG data collected by the one or more electrodes to identify a cardiac arrhythmia; and (ii) in response to identifying the cardiac arrhythmia, activate the pump to supply a cooled fluid from the fluid reservoir. The control system may also be programmed and operable to: (a) after activating the pump, further analyze the EKG data collected by the one or more electrodes to identify cessation of the cardiac arrhythmia; and (b) in response to identifying the cessation of the cardiac arrhythmia, deactivate the pump to cease the supply of the cooled fluid from the fluid reservoir. The medical device system may also include a steerable sheath configured contain the balloon in a deflated configuration and to percutaneously deliver the balloon to an epicardial space and/or epicardial surface. In some embodiments, the balloon comprises an hourglass shape. The balloon may include a doppler wire or electrodes by which a coronary artery can be detected using the control system. The balloon may include a fluid pressure sensor. The balloon may include at least one insulated portion that is more thermally insulated than other portions of the balloon.

In another aspect, this disclosure is directed to a method of treating cardiac arrhythmia using any embodiment of the medical device system described herein. The method includes: (1) during an open chest surgical procedure that exposes a heart of the patient, surgically placing the balloon in an epicardial space and/or on an epicardial surface of the heart; (2) identifying, by the control system, a cardiac arrhythmia by analyzing EKG data collected by one or more electrodes on the balloon and/or on the distal end portion of the fluid line; and (3) in response to identifying the cardiac arrhythmia, activating the pump to supply a cooled fluid from the fluid reservoir to the balloon so that the balloon cools the epicardial space and/or the epicardial surface.

Such a method of treating cardiac arrhythmia may optionally include one or more of the following features. The method may also include: after activating the pump, further analyzing the EKG data to identify cessation of the cardiac arrhythmia; and in response to identifying the cessation of the cardiac arrhythmia, deactivating the pump to cease the supply of the cooled fluid from the fluid reservoir to the balloon.

In another aspect, this disclosure is directed to another method of treating cardiac arrhythmia using any embodiment of the medical device system described herein. The method includes: (i) percutaneously navigating, within a patient, a steerable sheath containing the balloon in a deflated configuration; (ii) deploying the balloon from the sheath to an epicardial space and/or epicardial surface of a heart of the patient; (iii) identifying, by the control system, a cardiac arrhythmia by analyzing EKG data collected by one or more electrodes on the balloon and/or on the distal end portion of the fluid line; and (iv) in response to identifying the cardiac arrhythmia, activating the pump to supply a cooled fluid from the fluid reservoir to the balloon so that the balloon cools the epicardial space and/or the epicardial surface.

Such a method of treating cardiac arrhythmia may optionally include one or more of the following features. The method may also include: after activating the pump, further analyzing the EKG data to identify cessation of the cardiac arrhythmia; and in response to identifying the cessation of the cardiac arrhythmia, deactivating the pump to cease the supply of the cooled fluid from the fluid reservoir to the balloon. The method may also include percutaneously withdrawing the balloon from the epicardial space and/or the epicardial surface of the heart of the patient by positioning the balloon in the steerable sheath or another sheath.

Particular embodiments of the subject matter described in this document can be implemented to realize one or more of the following advantages. First, transient therapeutic tissue cooling therapy can be delivered using the devices and methods described herein. In some embodiments, heart conditions such as arrhythmias and others can be treated using the devices and methods provided herein. In some embodiments, arrhythmias can be treated relatively painlessly. In some embodiments, the upcoming onset of arrhythmias can be predicted and epicardial cooling therapy can be delivered to prevent the arrhythmias. In some cases, such conditions can be treated in a minimally invasive fashion using the devices and methods provided herein. Such minimally invasive techniques can reduce recovery times, patient discomfort, and treatment costs.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. In addition, the materials, methods and examples are illustrative only and not intended to be limiting.

The details of one or more embodiments of the invention are set forth in the accompanying drawings and the description below. Other aspects, features, and advantages of the invention will be apparent from the description, the drawings, and the claims.

### DESCRIPTION OF DRAWINGS

FIG. 1 depicts a patient using an example medical device system that treats cardiac arrhythmias.

FIG. 2 shows an example balloon cooling device of the medical device system of FIG. 1 in position on a heart of the patient.

FIG. 3 is a perspective view of the balloon cooling device of FIG. 2.

FIG. 4 shows another example balloon cooling device of the medical device system of FIG. 1 in position on a heart of the patient.

FIG. 5 is a flowchart of an example method of treating a cardiac arrhythmia using the medical device system of FIG. 1.

FIG. 6 shows an example of a generic computer device and a generic mobile computer device that may be used in connection with the devices and systems described in this specification.

Like reference numbers represent corresponding parts throughout.

### DETAILED DESCRIPTION

This document describes medical device systems and related methods for delivering therapy via temperature modulation. For example, this document describes medical device systems and related methods for the treatment of cardiac arrhythmias (e.g., atrial fibrillation) by delivering cooling to epicardial tissue.

In some embodiments, heart conditions, such as arrhythmias and others, can be treated using the devices, systems, and methods described herein. In some embodiments described herein, arrhythmias can be treated by a balloon-based system for painlessly terminating arrhythmias. The devices and methods provided herein permit prompt termination of atrial fibrillation almost immediately after an episode begins (to prevent persistence) and is effective irrespective of patient age and comorbidities.

This disclosure describes implantable devices that deliver non-destructive rapid and temporary cooling of tissues, such as cardiac tissues for the termination (and potentially prevention) of atrial arrhythmias. In some embodiments, the system is capable of temporarily cooling tissues in a controlled fashion by any desired amount, up to about 20 degrees Celsius (or colder in some cases, e.g., to about 5 degrees Celsius). The use of non-freezing cold is particularly attractive due to its well-established safety record in cardiac applications.

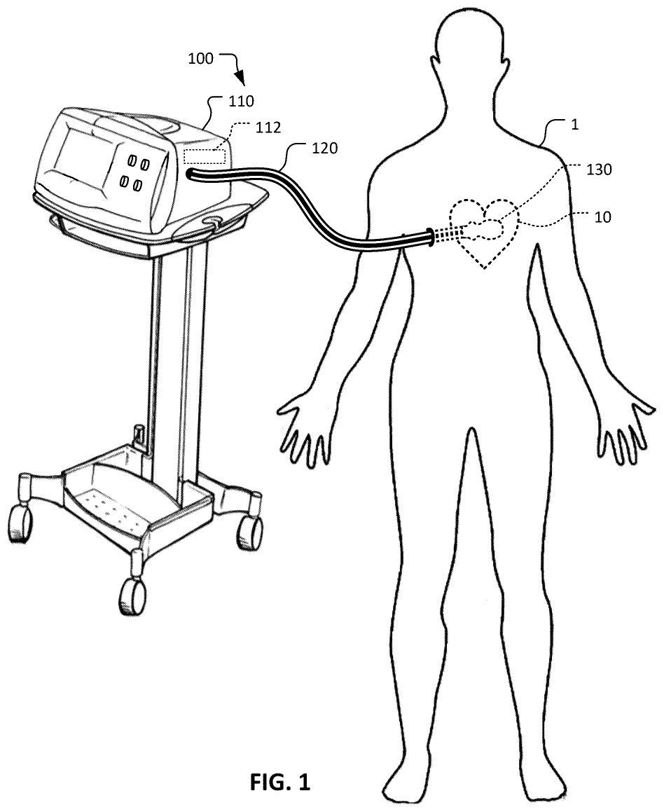

FIG. 1 depicts a patient 1 with a heart 10. The patient 1 is being treated by an example medical device system 100 in accordance with this disclosure. The medical device system 100 broadly includes console 110, a fluid line 120, and a balloon 130. The balloon 130 is implanted in the patient 1 in contact with, or proximate to, epicardial tissue of the heart 10. The fluid line 120 extends from the console 110 and percutaneously to the balloon 130. The fluid line 120 defines at least two lumens by which cooling fluid can be supplied from the console 110 to the balloon 130 and returned from the balloon 130 to the console 110.

The balloon 130 can be initially positioned in contact with, or proximate to, the epicardial tissue of the heart 10 in multiple ways. For example, in some cases the balloon 130 can be surgically positioned in contact with, or proximate to, the epicardial tissue of the heart 10 during an open-chest surgery (e.g., during a bypass graft surgery, valve repair/replacement, etc.). The fluid line 120 can be installed from the balloon 130 to the console 110 in a manner like that of a chest tube. In such a case, the medical device system 100 can be used temporarily to treat post-operative fibrillation (e.g., a-fib). For example, in such a case the medical device system 100 can be used during the first week after open heart surgery. After the treatment period, the balloon 130 can be percutaneously extracted/removed without the need for open surgery. In some cases, a retrieval and/or extraction sheath device can be used to percutaneously extract/remove the balloon 130. In some cases, a suction device can be used to remove fluid from the chest cavity, and/or to percutaneously extract/remove the balloon 130. Balloon deflation could also change its shape, facilitating removal.

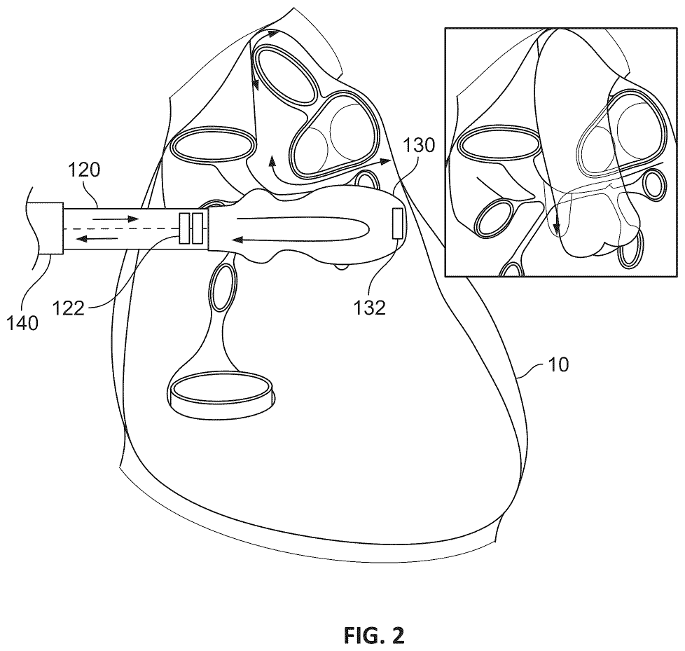

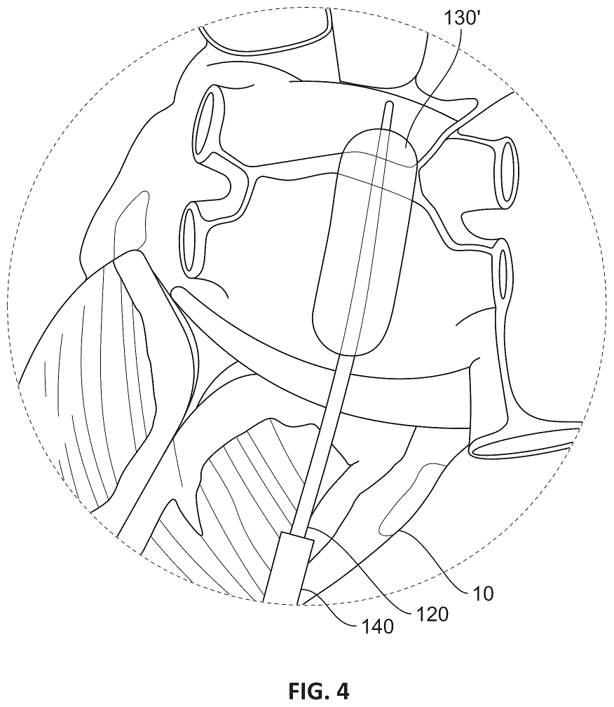

In another example, the balloon 130 can be percutaneously delivered in a minimally-invasive manner using a steerable sheath 140 (e.g., see FIGS. 2 and 4). In such a case, the balloon 130 can be contained in the steerable sheath 140 in a collapsed (deflated) configuration. In some embodiments, one or more radiopaque markers are included on the steerable sheath 140 and/or the balloon 130 to assist with the minimally-invasive process of placing the balloon 130 under an imaging modality (e.g., fluoroscopy, etc.).

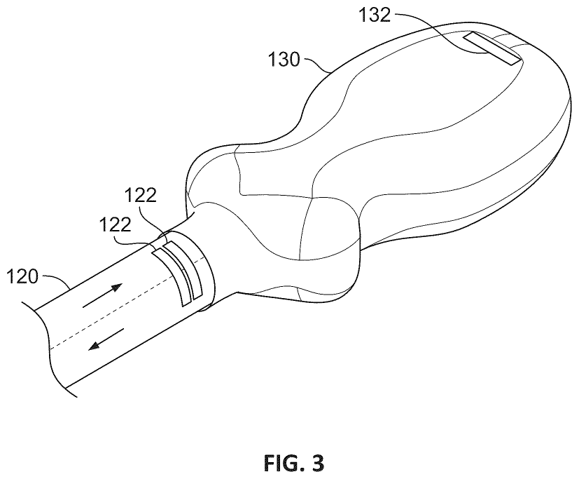

In some embodiments, the medical device system 100 can also include one or more electrodes 122/132 (FIGS. 2 and 3) that can be used to capture EKG signals/data from the heart 10. The EKG signals/data can be transmitted to the console 110 (e.g., via one or more electrical wires extending along the fluid line 120), and a control system 112 of the console 110 can process, monitor, assess the electrocardiogram (“EKG”) of the patient 1. In some embodiments, the one or more electrodes 122/132 can also be used to deliver pacing to the heart 10.

In some embodiments, another EKG system (e.g., with skin patch electrodes, not shown) can be used in communication with the console 110. In some such embodiments, the control system 112 of the console 110 can process, monitor, and assess the EKG of the patient 1 from such another EKG system.

In addition to the control system 112 of the console 110, the console 110 can include a pump, a fluid reservoir, and a fluid cooling system (e.g., chiller). The fluid reservoir can contain sterile fluid (e.g., sterile saline) and the fluid cooling system can keep the sterile fluid at a constant cooled temperature (e.g., at about 5° C., or in a range of about 1° C. to 5° C.). When the control system 112 of the console 110 detects fibrillation by analyzing the EKG signals, the control system 112 can activate the pump to circulate the cooled fluid to the balloon 130 via the dual lumen fluid line 120. The balloon 130 can thereby cool the epicardial tissue of the heart 10 to treat the fibrillation. When the EKG data indicates that the fibrillation has ceased, then control system 112 of the console 110 can then stop the pump to cease the cooling therapy delivered to the heart 10 via the balloon 130. It is also understood that the pressure of the balloon 130 can be continuously monitored in some embodiments. Such monitoring would permit assessment of balloon contact with atrial tissue. Additionally, since the intra-balloon pressure is affected by the left atrium, with which it is in contact, such a measurement can be used to calculate left atrial pressure (analogous to a wedge pressure), to provide hemodynamic monitoring without the need for intravascular lines (which have been associated with sepsis).

In some embodiments, the temperature of the balloon 130 during the cooling ranges from about 1 degree Celsius to about 37 degrees Celsius. In some embodiments, the therapeutic cold temperature of the balloon 130 ranges from about 5 degree Celsius to about 15 degrees Celsius. In some embodiments, the pump and control system 112 of the console 110 can control/modulate the flowrate of the cooling fluid to achieve and maintain a prespecified epicardial tissue temperature (or temperature range).

In some embodiments, the cooling therapy is delivered until the fibrillation ceases. In some embodiments, a time-based cooling therapy is delivered and the therapeutic period of time during which cooling is delivered ranges from about 5 seconds to about 180 seconds.

The control system 112 of the control module of the console 110 can include several programmable parameters such as the cooling temperature set point and a duration of the cooling therapy. Programmable parameters of the cooling therapy may include a cooling temperature set point (e.g., about 5 degrees Celsius to about 15 degrees Celsius), a duration (e.g., on time) (e.g., about 5 seconds to about 180 seconds), a therapy target cooling temperature range (e.g., a minimum and a maximum therapeutic target temperature) (e.g., about 15 degrees Celsius to about 5 degrees Celsius), a maximum (e.g., a maximum threshold) cool temperature (corresponding to the cold surface of the balloon 130) (e.g., about 5 degrees Celsius to about 0 degrees Celsius). A safety termination of cooling therapy (e.g., the pump of the console 110 is turned off) may result if one of the following conditions is met: the maximum cold threshold temperature is exceeded (corresponding to the cold surface of the housing portion 110b) (e.g., about 5 degrees Celsius to about 0 degrees Celsius).

Also referring to FIGS. 2 and 3, in some embodiments the balloon 130 can have an hourglass shape and/or can be placed adjacent to the heart 10 (e.g., in an oblique sinus region of the heart 10). Such an hourglass shape of the balloon 130 can shape the balloon 130 to match the oblique sinus. In some embodiments, the balloon 130 can be placed in the coronary sinus region, left atrium, left ventricle, or elsewhere in contact with the heart 10.

In some embodiments, the balloon 130 comprises one or more fixation elements configured to secure the balloon 130 to adjacent tissue. The one or more fixation elements can be barbs, helical elements, roughened surface portions, tines, hooks, and the like, and combinations thereof.

Referring to FIG. 4, in some example embodiments the balloon 130′ can have a cylindrical shape and can be positioned in an oblique sinus.

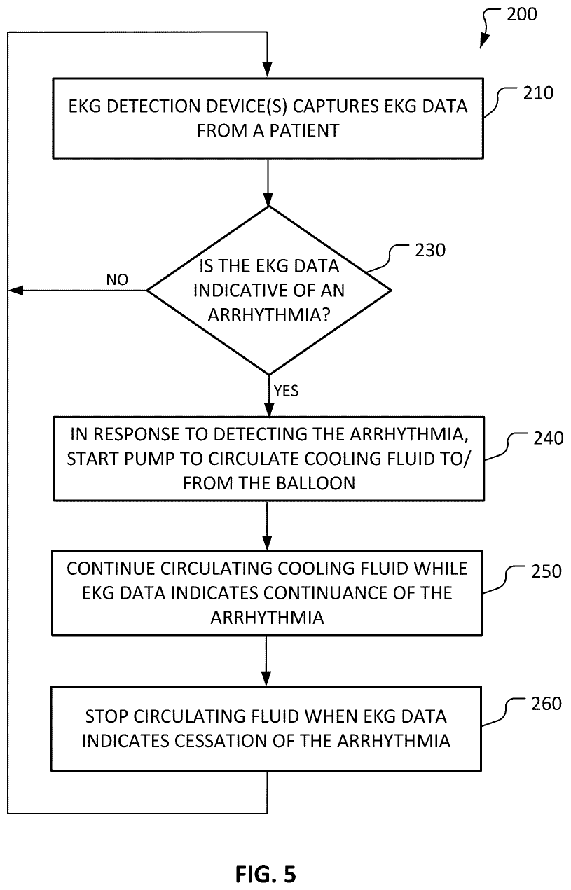

FIG. 5 is a flowchart of a method 200 for treating a cardiac arrhythmia in a patient in need thereof, in accordance with some embodiments provided herein. In some embodiments, the method 200 can be performed by, or using, the implantable medical device system 100 described herein.

In step 210, EKG data from the patient (e.g., using the one or more electrodes 122/132, using skin patch electrodes, and/or using an external heart monitoring system) is captured by the control system 112 of the console 110.

In step 230, the EKG data captured from the patient is analyzed to determine whether the EKG data is indicative of an occurring cardiac arrhythmia or predictive of an upcoming onset of a cardiac arrhythmia. The control system 112 of the console 110 and/or an external controller device can run one or more algorithms to analyze the captured EKG data. The algorithms can be designed to identify one or more types of an irregular pattern of the received EKG data. That is, the one or more algorithms can be used to determine whether the EKG data captured by the EKG detection device indicates that the patient is experiencing, or will soon experience, one or more types of cardiac arrhythmia.

In step 240, in response to a detection by the control system 112 of the console 110 and/or by an external controller device of a heart monitoring system that the patient is experiencing, or will soon experience, a cardiac arrhythmia, the control system 112 of the console 110 can start the pump to circulate cooling fluid to the balloon 130 via the fluid line 120. In some embodiments, the control system 112 of the console 110 can also emit an alert or an alarm to indicate that a cardiac arrhythmia is occurring. The alert/alarm can be audible, tactile, visual, and/or combinations thereof. In some embodiments, an alert/alarm can be sent to one or more remote receivers via Wi-Fi or a cell phone transmission. The remote receivers can be that of a caretaker, physician, clinic, healthcare provider, and the like.

In some embodiments, step 240 also includes (in addition to the detection of the arrhythmia) an analysis by the patient control device to determine whether cooling therapy should be delivered in response to the detection of the arrhythmia. Such analysis can be based on factors such as, but not limited to, the type of alarm/alert, the EKG data, one or more threshold parameters (which can be customized for the particular patient), patient history, and the like.

In some embodiments, the alert emitted by the external patient control device can include information regarding the detection of the arrhythmia (e.g., displayed on a display device of the console 110). The display device (or other aspects of the user interface of the console 110) can also facilitate user input to the console 110. For example, in some embodiments the user can enter a selection to indicate whether or not the user desires the cooling therapy to be delivered.

In step 250, the circulation of the cooling fluid is continued while the EKG data continues to indicate the occurrence of prediction of fibrillation.

In step 260, when the captured EKG data is no longer indicative of a cardiac arrhythmia, the control system 112 of the console 110 can stop delivering power to the pump so that the supply of the cooling fluid to the balloon 130 is ceased and the balloon 130 stops cooling the epicardial tissue of the patient.

### Additional Optional Features of the Medical Device System Described Herein

In some embodiments provided herein, the balloon 130 can include temperature monitoring devices (e.g., integrated thermocouples, thermistors, or other types of temperature monitoring devices) for temperature registration and feedback.

In some embodiments, the balloon 130 can include pressure monitoring devices for the console 110 to monitor and/or control the fluid pressure within the balloon 130.

In some embodiments, the balloon 130 can be affixed to a non-biodegradable fabric to permit surgical suturing of balloon 130 to target tissues and/or to increase friction with adjacent tissues.

In some embodiments, the one or more electrodes 122/132 on the cooling balloon 130 and/or on the fluid line 120 continuously record local atrial and ventricular electrograms. In some embodiments, simultaneous surface ECG electrodes can also be part of the system 100. These may be true surface or subcutaneous electrodes, or a mix depending on position. In some embodiments, post op data collection will serve as the training set for supervised AF detection, with a modest data set size required by means of transfer learning and foundational models. In some embodiments, an AI model run by the control system 112 of the console 110 would use signals received from the one or more electrodes 122/132 on the cooling balloon 130 and/or on the fluid line 120 and the surface electrode ECG signals to predict fibrillation before its onset, and to then initiate epicardial cooling via the balloon 130 to prevent the onset of fibrillation.

In some embodiments, the system 100 can include a doppler wire on the balloon 130. The doppler wire (or electrodes) can be used to identify the presence of an adjacent coronary artery, and/or can be used to help identify fibrillation.

In some embodiments, one or more portions of the balloon 130 are more thermally insulated in comparison to other portions of the balloon 130.

In some embodiments, the balloon 130 can be withdrawn from the patient using a sheath to encompass the balloon within the sheath, and then removing. In some embodiments, the balloon 130 can be withdrawn from the patient without a sheath.

In some embodiments, the cooling module is patient activated. In some embodiments, the cooling module is activated automatically if one or more conditions is/are detected (e.g., by sensors in communication with the balloon 130 and/or the console 110) that indicate that providing tissue cooling will be beneficial to the patient.

In some embodiments, pacing (from the electrodes 122/132) is delivered to the heart concurrently with delivery of the cooling therapy.

In some embodiments, the cooling fluid drains out of the balloon 130 after cessation of the cooling therapy.

In some embodiments, the console 110 can include a display screen. The console 110 can communicate with an existing ECG system. The console 110 can capture data from the ECG system and display the data on the display screen.

In some embodiments, the control system 112 of the console 110 can be software upgradeable. The control system 112 can be configured and/or programmed with the ability to sense, detect and alert to arrhythmias independently. In some embodiments, the control system 112 can be configured and/or programmed with the ability to use artificial intelligence (“AI”) to predict onset of fibrillation, and to commence cooling treatment in response thereto.

In some embodiments, a suture sleeve is included on the fluid line 120 to connect the fluid line 120 to the skin of the patient 1 following placement.

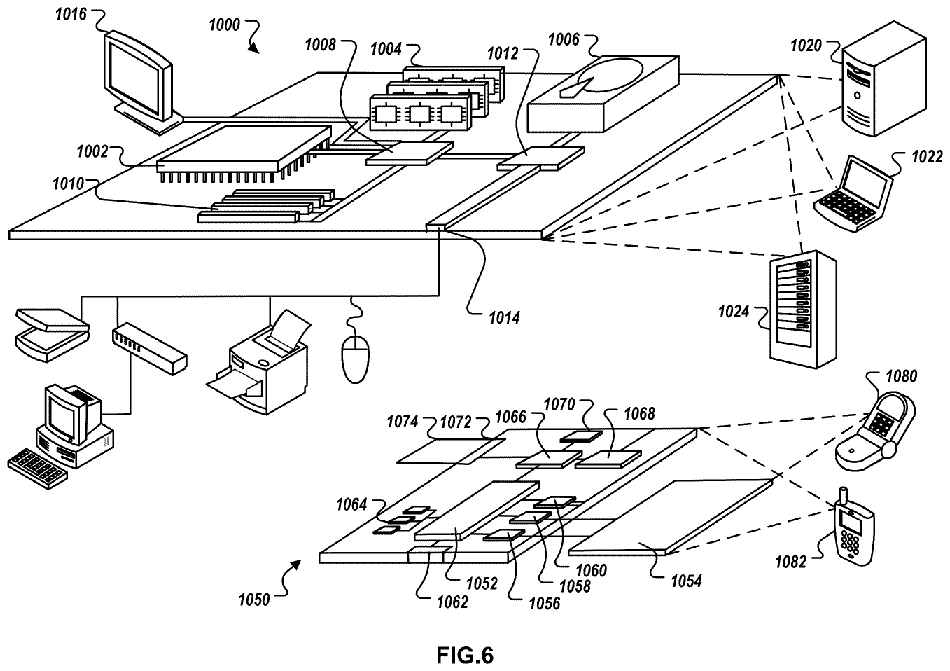

FIG. 6 is a block diagram of computing devices 1000, 1050 that may be used to implement the systems (e.g., the control system 112 of the console 110) and methods described in this document, as either a client or as a server or plurality of servers. Computing device 1000 is intended to represent various forms of digital computers, such as laptops, desktops, workstations, personal digital assistants, servers, blade servers, mainframes, and other appropriate computers. Computing device 1050 is intended to represent various forms of mobile devices, such as personal digital assistants, cellular telephones, smartphones, and other similar computing devices. Additionally computing device 1000 or 1050 can include Universal Serial Bus (USB) flash drives. The USB flash drives may store operating systems and other applications. The USB flash drives can include input/output components, such as a wireless transmitter or USB connector that may be inserted into a USB port of another computing device. The components shown here, their connections and relationships, and their functions, are meant to be exemplary only, and are not meant to limit implementations described and/or claimed in this document.

Computing device 1000 includes a processor 1002, memory 1004, a storage device 1006, a high-speed interface 1008 connecting to memory 1004 and high-speed expansion ports 1010, and a low speed interface 1012 connecting to low speed bus 1014 and storage device 1006. Each of the components 1002, 1004, 1006, 1008, 1010, and 1012, are interconnected using various busses, and may be mounted on a common motherboard or in other manners as appropriate. The processor 1002 can process instructions for execution within the computing device 1000, including instructions stored in the memory 1004 or on the storage device 1006 to display graphical information for a GUI on an external input/output device, such as display 1016 coupled to high speed interface 1008. In other implementations, multiple processors and/or multiple buses may be used, as appropriate, along with multiple memories and types of memory. Also, multiple computing devices 1000 may be connected, with each device providing portions of the necessary operations (e.g., as a server bank, a group of blade servers, or a multi-processor system).

The memory 1004 stores information within the computing device 1000. In one implementation, the memory 1004 is a volatile memory unit or units. In another implementation, the memory 1004 is a non-volatile memory unit or units. The memory 1004 may also be another form of computer-readable medium, such as a magnetic or optical disk.

The storage device 1006 is capable of providing mass storage for the computing device 1000. In one implementation, the storage device 1006 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 1004, the storage device 1006, or memory on processor 1002.

The high speed controller 1008 manages bandwidth-intensive operations for the computing device 1000, while the low speed controller 1012 manages lower bandwidth-intensive operations. Such allocation of functions is exemplary only. In one implementation, the high-speed controller 1008 is coupled to memory 1004, display 1016 (e.g., through a graphics processor or accelerator), and to high-speed expansion ports 1010, which may accept various expansion cards (not shown). In the implementation, low-speed controller 1012 is coupled to storage device 1006 and low-speed expansion port 1014. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 1000 may be implemented in a number of different forms, as shown in the figure. For example, it may be implemented as a standard server 1020, or multiple times in a group of such servers. It may also be implemented as part of a rack server system 1024. In addition, it may be implemented in a personal computer such as a laptop computer 1022. Alternatively, components from computing device 1000 may be combined with other components in a mobile device (not shown), such as device 1050. Each of such devices may contain one or more of computing device 1000, 1050, and an entire system may be made up of multiple computing devices 1000, 1050 communicating with each other.

Computing device 1050 includes a processor 1052, memory 1064, an input/output device such as a display 1054, a communication interface 1066, and a transceiver 1068, among other components. The device 1050 may also be provided with a storage device, such as a microdrive or other device, to provide additional storage. Each of the components 1050, 1052, 1064, 1054, 1066, and 1068, are interconnected using various buses, and several of the components may be mounted on a common motherboard or in other manners as appropriate.

The processor 1052 can execute instructions within the computing device 1050, including instructions stored in the memory 1064. The processor may be implemented as a chipset of chips that include separate and multiple analog and digital processors. Additionally, the processor may be implemented using any of a number of architectures. For example, the processor 1052 may be a CISC (Complex Instruction Set Computers) processor, a RISC (Reduced Instruction Set Computer) processor, or a MISC (Minimal Instruction Set Computer) processor. The processor may provide, for example, for coordination of the other components of the device 1050, such as control of user interfaces, applications run by device 1050, and wireless communication by device 1050.

Processor 1052 may communicate with a user through control interface 1058 and display interface 1056 coupled to a display 1054. The display 1054 may be, for example, a TFT (Thin-Film-Transistor Liquid Crystal Display) display or an OLED (Organic Light Emitting Diode) display, or other appropriate display technology. The display interface 1056 may comprise appropriate circuitry for driving the display 1054 to present graphical and other information to a user. The control interface 1058 may receive commands from a user and convert them for submission to the processor 1052. In addition, an external interface 1062 may be provide in communication with processor 1052, so as to enable near area communication of device 1050 with other devices. External interface 1062 may provide, for example, for wired communication in some implementations, or for wireless communication in other implementations, and multiple interfaces may also be used.

The memory 1064 stores information within the computing device 1050. The memory 1064 can be implemented as one or more of a computer-readable medium or media, a volatile memory unit or units, or a non-volatile memory unit or units. Expansion memory 1074 may also be provided and connected to device 1050 through expansion interface 1072, which may include, for example, a SIMM (Single In Line Memory Module) card interface. Such expansion memory 1074 may provide extra storage space for device 1050, or may also store applications or other information for device 1050. Specifically, expansion memory 1074 may include instructions to carry out or supplement the processes described above, and may include secure information also. Thus, for example, expansion memory 1074 may be provide as a security module for device 1050, and may be programmed with instructions that permit secure use of device 1050. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 1064, expansion memory 1074, or memory on processor 1052 that may be received, for example, over transceiver 1068 or external interface 1062.

Device 1050 may communicate wirelessly through communication interface 1066, which may include digital signal processing circuitry where necessary.

Communication interface 1066 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 1068. In addition, short-range communication may occur, such as using a Bluetooth, WiFi, or other such transceiver (not shown). In addition, GPS (Global Positioning System) receiver module 1070 may provide additional navigation- and location-related wireless data to device 1050, which may be used as appropriate by applications running on device 1050.

Device 1050 may also communicate audibly using audio codec 1060, which may receive spoken information from a user and convert it to usable digital information. Audio codec 1060 may likewise generate audible sound for a user, such as through a speaker, e.g., in a handset of device 1050. Such sound may include sound from voice telephone calls, may include recorded sound (e.g., voice messages, music files, etc.) and may also include sound generated by applications operating on device 1050.

The computing device 1050 may be implemented in a number of different forms, as shown in the figure. For example, it may be implemented as a cellular telephone 1080. It may also be implemented as part of a smartphone 1082, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system may include clients and servers. A client and server are generally remote from each other and typically interact through a communication network. The relationship of client and server arises by virtue of computer programs running on the respective computers and having a client-server relationship to each other.

A number of embodiments have been described. Nevertheless, it will be understood that various modifications may be made without departing from the spirit and scope of the invention. In addition, the logic flows depicted in the figures do not require the particular order shown, or sequential order, to achieve desirable results. In addition, other steps may be provided, or steps may be eliminated, from the described flows, and other components may be added to, or removed from, the described systems.

Accordingly, other embodiments are within the scope of the following claims.

What is claimed is:

1. A medical device system comprising:
a console comprising:
a control system;
a pump;
a fluid reservoir; and
a fluid cooling system;
a fluid line defining a fluid supply lumen and a fluid return lumen; and
a balloon attached or attachable at a distal end portion of the fluid line.

2. The medical device system of claim 1, further comprising one or more electrodes coupled to the balloon or the distal end portion of the fluid line.

3. The medical device system of claim 2, further comprising one or more electrical wires that are: (i) extending along the fluid line, (ii) connected or connectable to the one or more electrodes, and (iii) connected or connectable to the control system.

4. The medical device system of claim 3, wherein the control system is programmed and operable to:
analyze EKG data collected by the one or more electrodes to identify a cardiac arrhythmia; and
in response to identifying the cardiac arrhythmia, activate the pump to supply a cooled fluid from the fluid reservoir.

5. The medical device system of claim 4, wherein the control system is further programmed and operable to:
after activating the pump, further analyze the EKG data collected by the one or more electrodes to identify cessation of the cardiac arrhythmia; and
in response to identifying the cessation of the cardiac arrhythmia, deactivate the pump to cease the supply of the cooled fluid from the fluid reservoir.

6. The medical device system of claim 1, further comprising a steerable sheath configured contain the balloon in a deflated configuration and to percutaneously deliver the balloon to an epicardial space and/or epicardial surface.

7. The medical device system of claim 1, wherein the balloon comprises an hourglass shape.

8. The medical device system of claim 1, wherein the balloon comprises a doppler wire or electrodes by which a coronary artery can be detected using the control system.

9. The medical device system of claim 1, wherein the balloon comprises a fluid pressure sensor.

10. The medical device system of claim 1, wherein the balloon comprises at least one insulated portion that is more thermally insulated than other portions of the balloon.

11. A method of treating cardiac arrhythmia using the medical device system of claim 1, the method comprising:
during an open chest surgical procedure that exposes a heart of the patient, surgically placing the balloon in an epicardial space and/or on an epicardial surface of the heart;
identifying, by the control system, a cardiac arrhythmia by analyzing EKG data collected by one or more electrodes on the balloon and/or on the distal end portion of the fluid line; and
in response to identifying the cardiac arrhythmia, activating the pump to supply a cooled fluid from the fluid reservoir to the balloon so that the balloon cools the epicardial space and/or the epicardial surface.

12. The method of claim 11, further comprising:
after activating the pump, further analyzing the EKG data to identify cessation of the cardiac arrhythmia; and
in response to identifying the cessation of the cardiac arrhythmia, deactivating the pump to cease the supply of the cooled fluid from the fluid reservoir to the balloon.

13. A method of treating cardiac arrhythmia using the medical device system of claim 1, the method comprising:
percutaneously navigating, within a patient, a steerable sheath containing the balloon in a deflated configuration;
deploying the balloon from the sheath to an epicardial space and/or epicardial surface of a heart of the patient;
identifying, by the control system, a cardiac arrhythmia by analyzing EKG data collected by one or more electrodes on the balloon and/or on the distal end portion of the fluid line; and
in response to identifying the cardiac arrhythmia, activating the pump to supply a cooled fluid from the fluid reservoir to the balloon so that the balloon cools the epicardial space and/or the epicardial surface.

14. The method of claim 13, further comprising:
after activating the pump, further analyzing the EKG data to identify cessation of the cardiac arrhythmia; and
in response to identifying the cessation of the cardiac arrhythmia, deactivating the pump to cease the supply of the cooled fluid from the fluid reservoir to the balloon.

15. The method of claim 13, further comprising percutaneously withdrawing the balloon from the epicardial space and/or the epicardial surface of the heart of the patient by positioning the balloon in the steerable sheath or another sheath.
